(19) United States
(12) Patent Application Publication
(10) Pub. No.: US 2014/0263722 A1
(43) Pub. Date: Sep. 18, 2014

(54) AROMATIC NEBULIZING DIFFUSER

(71) Applicant: SERENE HOUSE INTERNATIONAL ENTERPRISE LTD., Tortola (VG)

(72) Inventor: Ming Jen Hsiao, Miaoli (TW)

(73) Assignee: Serene House International Enterprise Ltd., Tortola (VG)

(21) Appl. No.: 13/839,419

(22) Filed: Mar. 15, 2013

(51) Int. Cl. A61L 9/14; B05B 17/06

(52) U.S. Cl. CPC A61L 9/14; B05B 17/0615
USPC 239/102.2

(57) ABSTRACT

An aromatic nebulizing diffuser includes a bottom holder shell, an electric fan, a fluid container, an oscillator, a circuit board, a power supply unit and a cover shell. The oscillator is adapted to oscillate an aromatic fluid in the fluid container into a mist of aromatic fluid droplets. The fluid container has a side block defining an air passage for guiding electric fan induced air into a pressurized space in between the cover shell and the fluid container to enhance the effects of mist diffusion. A rotating connection structure is provided between the bottom holder shell and the cover shell, facilitating general maintenance and cleaning activities.

### BACKGROUND OF THE INVENTION

1. Field of the Invention

The present invention relates to an aromatic nebulizing diffuser and more particularly, to such an aromatic nebulizing diffuser, which provides maximized aromatic fluid holding capacity, creates enhanced decorative and visual effects of aroma diffusion, and facilitates general maintenance and cleaning activities.

2. Description of the Related Art

With fast economic development and technological advancement and the rise of human civilization, there is a growing emphasis on the level of material and spiritual life, and having a good living environment has become the goal of people actively want to pursue. Further, air conditioners, air purifiers, fragrances, electric jasmines, aromatic nebulizing diffusers, perfume dispensers are intensively used in offices, rooms, cars, and many other living environments to eliminate bad smells and stale air, improving the work efficiency and mental health. Further, many new designs of aromatic nebulizing diffusers and perfume dispensers have been continuously created and have appeared on the market.

However, commercial aromatic nebulizing diffusers and perfume dispensers commonly have the following drawbacks:
1. Because the air guide conduct is mounted in the fluid container for guiding outside air into the fluid container, its size is limited, lower the effect of pressurizing the intake flow of air. Further, the presence of the air guide conduct in the fluid container gives resistance to the intake flow of air, lowering the aroma diffusing effect.
2. The air guide conduct occupies much inside space of the fluid container, reducing the aromatic fluid holding capacity of the fluid container.
3. The spout or nozzle design is simply for output of created mist of aromatic fluid droplets, it cannot create enhanced decorative and visual effects of aroma diffusion.
4. After a long use of an aromatic nebulizing diffuser or perfume dispenser, the internal components may be covered with oil stains. However, the complicated component mounting structure of a conventional aromatic nebulizing diffuser or perfume dispenser does not facilitate general maintenance and cleaning activities.

Therefore, it is desirable to provide an aromatic nebulizing diffuser, which eliminates the aforesaid drawbacks.

### SUMMARY OF THE INVENTION

The present invention has been accomplished under the circumstances in view. It is main object of the present invention to provide an aromatic nebulizing diffuser, which provides enhanced decorative and visual effects of aroma diffusion.

It is another object of the present invention to provide an aromatic nebulizing diffuser, which maximizes the fluid accommodation capacity.

It is still another object of the present invention to provide an aromatic nebulizing diffuser, which facilitates general maintenance and cleaning activities.

To achieve this and other objects of the present invention, an aromatic nebulizing diffuser comprises a bottom holder shell, which comprises an accommodation open chamber and a plurality of air vents in communication with the accommodation open chamber, an electric fan mounted in the accommodation open chamber and operable to suck outside air through the air vents into the accommodation open chamber, a fluid container, which comprises a container body mounted in the bottom holder shell to close the accommodation open chamber and adapted for holding an aromatic fluid, a through hole located at the bottom side of the container body, a side block located at the periphery of the container body and defining a bottom air inlet in air communication with the accommodation open chamber and air vents of the bottom holder shell, a top air outlet and an air passage in communication between the bottom air inlet and the top air outlet, a container cover detachably covering the container body, an air intake port and a coupling tube located at the container body in air communication with the inside space of the container body, an oscillator sealed in the through hole of the fluid container and adapted to oscillate an aromatic fluid in the container body into a mist of aromatic fluid droplets, a circuit board electrically coupled with the oscillator, a power supply unit electrically connected to the electric fan, the oscillator and the circuit board to provide the electric fan, the oscillator and the circuit board with the necessary working voltage, and a cover shell, which comprises a bottom open end coupled to the bottom holder shell to surround the fluid container, a pressurized space defined therein around the fluid container and an inside coupling hole coupled to the coupling tube.

Preferably, the bottom holder shell comprises a plurality of columns and a step located on the inside thereof; the container body comprises a bottom mounting flange supported on the step of the bottom holder shell and affixed to the columns with fastening members.

Preferably, a gasket ring is sealed in between the bottom mounting flange of the container body and the step of the bottom holder shell.

Preferably, the container cover comprises a plurality of locating rods; the cover shell comprises a plurality of locating sleeves respectively coupled to the locating rods of the container cover.

Preferably, the aromatic nebulizing diffuser further comprises an oscillator sealing gasket ring mounted in the through hole of the fluid container to seal the oscillator in the through hole, and a locating plate bonded to the container body of the fluid container to seal the oscillator sealing gasket ring in the through hole of the fluid.

Preferably, the cover shell further comprises a flat ramp-shaped spout located at the top side thereof in communication with the coupling hole for output of an oscillated mist of aromatic fluid droplets.

Preferably, the aromatic nebulizing diffuser further comprises a plurality of elastic mounting members mounted in the accommodation open chamber of the bottom, and a plurality of light-emitting devices mounted in the elastic mounting members and electrically connected to the power supply unit.

Preferably, the bottom holder shell and the cover shell are selectively made in one of transparent, translucent, partially transparent, locally transparent, partially translucent and locally translucent forms.

Preferably, the aromatic nebulizing diffuser further comprises a functional keypad mounted in the accommodation open chamber of the bottom holder shell. The functional keypad comprises a circuit board electrically coupled to the oscillator, the oscillator circuit board and the power supply unit, a plurality of key switches electrically connected to the circuit board for functional controls, and a plurality of light guide rods; the bottom holder shell comprises means for receiving the key switches and the light guide rods.

Preferably, the bottom holder shell comprises a plurality of retaining studs protruded from the inner side of the top edge thereof inside the accommodation open chamber; the cover shell comprises a bottom coupling flange downwardly extended from the bottom side thereof and insertable into the accommodation open chamber of the bottom holder shell, and a plurality of L-shaped retaining grooves located at said bottom coupling flange corresponding to the retaining studs. The L-shaped retaining grooves are respectively coupled to the retaining studs when the bottom coupling flange of the cover shell is vertically downwardly into the accommodation open chamber of the bottom holder shell and then rotated horizontally relative to the bottom holder shell through a predetermined angle.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of an aromatic nebulizing diffuser in accordance with the present invention.

FIG. 2 is an exploded view of the aromatic nebulizing diffuser in accordance with the present invention.

FIG. 3 is a sectional side view of the aromatic nebulizing diffuser in accordance with the present invention.

FIG. 4 is a sectional elevation of the aromatic nebulizing diffuser in accordance with the present invention.

FIG. 5 is another sectional elevation of the aromatic nebulizing diffuser in accordance with the present invention.

FIG. 6-1 is an applied view of the present invention, illustrating the operation of the rotating connection structure between the bottom holder shell and the cover shell (I).

FIG. 6-2 is an applied view of the present invention, illustrating the operation of the rotating connection structure between the bottom holder shell and the cover shell (II).

FIG. 6-3 is an applied view of the present invention, illustrating the operation of the rotating connection structure between the bottom holder shell and the cover shell (III).

FIG. 6-4 is an applied view of the present invention, illustrating the operation of the rotating connection structure between the bottom holder shell and the cover shell (IV).

### DETAILED DESCRIPTION OF THE PREFERRED EMBODIMENT

Referring to FIGS. 1 and 2, an aromatic nebulizing diffuser in accordance with the present invention is shown. The aromatic nebulizing diffuser comprises a bottom holder shell 10, an electric fan 20, a fluid container 30, an oscillator 40, an oscillator circuit board 50, a power supply unit 60, and a cover shell 70. The oscillator 40 is controlled to oscillate an aromatic fluid in the fluid container 30 into a mist of aromatic fluid droplets, which is then forced out of the fluid container 30 into the outside open air by a pressure produced in a pressurized space between the fluid container 30 and the cover shell 70, enhancing the effect of aroma diffusion. Further, the cover shell 70 provides a flat ramp-shaped spout for output of the generated mist of aromatic fluid droplets, enhancing decorative and visual effects. Further, the bottom holder shell 10 and the cover shell 70 are detachably fastened together by means of a rotating connection structure, facilitating general maintenance and cleaning activities.

Referring to FIG. 3 and FIG. 2 again, the bottom holder shell 10 comprises an accommodation open chamber 11, a plurality of air vents 12 located on the bottom side thereof in communication between the accommodation open chamber 11 and the atmosphere for taking in outside air, a plurality of columns 13 and a step 14 disposed inside the accommodation open chamber 11, and a plurality of foot members 15 fixedly mounted at the bottom side thereof and respectively equipped with a rubber pad 151 for supporting the bottom holder shell 10 stably on a flat surface.

Referring to FIG. 4 and FIG. 2 again, the electric fan 20 is mounted in the accommodation open chamber 11 and operable to suck outside air through the air vents 12 into the inside of the accommodation open chamber 11.

Referring to FIGS. 2-4 again, the fluid container 30 comprises a container body 31 adapted to hold an aromatic fluid (for example, a mixture of an essential oil and water), and a container cover 32 for covering the container body 31. The container body 31 comprises a bottom mounting flange 311 extending horizontally outwardly around the periphery of the bottom side thereof and affixed to the columns 13 of the bottom holder shell 10 with fastening members, for example, screws 312, a gasket ring 313 tightly sealed between the bottom mounting flange 311 and the step 14 to keep the accommodation open chamber 11 in an enclosed tight, a through hole 314 located on the bottom side thereof and adapted to accommodate the oscillator 40, a side block 315 located at the periphery thereof and upwardly extended from the bottom mounting flange 311, an air inlet 3161 located in the bottom mounting flange 311 at the bottom side of the side block 315, an air outlet 3162 located near the top side of the side block 315, and an air passage 316 defined in the side block 315 in communication between the air inlet 3161 and the air outlet 3162.

The container cover 32 is detachably covered on the container body 31 to facilitating filling an aromatic fluid in the container body 31, comprising an air intake port 321 in communication with the inside space of the container body 31, and a coupling tube 322 and a plurality of locating rods 323 for securing the cover shell 70.

Referring to FIGS. 2 and 3 again, the oscillator 40 is mounted within a gasket ring 41 in the through hole 314 of the container body 31, and then a locating plate 42 is affixed to the bottom side of the container body 31 to seal the through hole 314 and to hold the oscillator 40 in the through hole 314 within the gasket ring 41. Thus, the oscillator 40 can be operated to oscillate the aromatic fluid in the container body 31 into a mist of aromatic fluid droplets.

The oscillator circuit board 50 is mounted in the accommodation open chamber 11 and electrically connected to the oscillator 40 for driving the oscillator 40 to oscillate the aromatic fluid in the container body 31 into a mist of aerosol droplets.

Referring to FIG. 2 again, the power supply unit 60 is mounted in the accommodation open chamber 11 and electrically connected to the electric fan 20, the oscillator 40 and the oscillator circuit board 50. Preferably, the bottom holder shell 10 is preferably equipped with a port for enabling an external power supply to be connected to the topmost edge 101 of the power supply unit 60 and then provided by the power supply unit 60 to the electric fan 20, the oscillator 40 and the oscillator circuit board 50.

Referring to FIG. 5 and FIGS. 2-3 again, the cover shell 70 comprises a bottom open end 71 attached to the top edge 101 of the bottom holder shell 10 for enabling the cover shell 70 to surround the fluid container 30, and a coupling hole 72 coupled to the coupling tube 322. Thus, a pressurized space S is defined between the outer surface of the fluid container 30 and the inner surface of the cover shell 70.

The cover shell 70 further comprises a plurality of locating sleeves 73 respectively sleeved onto the locating rods 323 of the container cover 32. Subject to coupling between the locating sleeves 73 and the locating rods 323, the cover shell 70 can be quickly secured to or removed from the container cover 32. After removal of the cover shell 70 from the container cover 32, the user can open the container cover 32 from the container body 31 and fill an aromatic fluid in the container body 31.

The cover shell 70 further comprises a flat ramp-shaped spout 75 located at the top side thereof in communication with the coupling hole 72, the coupling tube 322 of the container cover 32 and the inside space of the container body 31.

Referring to FIGS. 1 and 2 again, elastic mounting members 81; 82 are mounted in the accommodation open chamber 11 of the bottom holder shell 10 to hold light-emitting devices 80 therein for giving off color light. The elastic mounting members 81; 82 can be made out of, for example, silicon rubber. The light-emitting devices 80 can be, for example, light-emitting diodes (LEDs) of different colors.

Further, the bottom holder shell 10 and the cover shell 70 can be transparent, translucent, partially or locally transparent, or partially or locally translucent. They can be selected from the material groups of glass, translucent ceramic, frosted glass, acrylic, plastic and crystal. Further, the bottom holder shell 10 and the cover shell 70 can be made in any of a variety of colors, or having any of a variety of color designs. By means of controlling the light-emitting devices 80 to give off colored light during diffusing of a mist of aromatic fluid droplets, the invention can create brilliant visual effects.

Referring to FIGS. 1, 2 and 5 again, the invention further comprises a functional keypad 90 mounted in the accommodation open chamber 11 of the bottom holder shell 10. The functional keypad 90 comprises a circuit board 91 electrically coupled to the oscillator 40, the oscillator circuit board 50 and the power supply unit 60, a plurality of key switches 92 electrically connected to the circuit board 91 for situational lighting options, time setting, power control and other related functional controls, and a plurality of light guide rods 93. The bottom holder shell 10 has means, for example, holes for receiving the key switches 92 and the light guide rods 93.

Referring to FIGS. 3 and 4 again, after understanding of the component parts of the aromatic nebulizing diffuser and their positioning, the operation and features of the aromatic nebulizing diffuser will now be outlined hereinafter. At first, the user needs to fill an aromatic fluid (for example, an essential oil and water mixture) into the container body 31 of the fluid container 30. Thereafter, the user can turn on the oscillator 40 and the electric fan 20, causing the oscillator 40 to oscillate the aromatic fluid into a mist of aromatic fluid droplets and the electric fan 20 to suck outside air through the air vent 12 of the bottom holder shell 10, the air inlet 3161, the air passage 316 and the air outlet 3162 into the pressurized space S, enabling the intake flow of air to go through the air intake port 321 of the container cover 32 into the container body 31 to force the diffused mist of aromatic fluid droplets out of the coupling hole 72 and flat ramp-shaped spout 75 of the cover shell 70 into the outside open air.

Because the dimension of the pressurized space S is larger than the diameter of the air intake port 321 of the container cover 32, subject to the principles of fluid dynamics, the intake flow of air induced by the electric fan 20 will be accumulated in the pressurized space S and pressurized, and then guided out of the air intake port 321 rapidly to form a strong upward airflow that will go through the coupling hole 72 and flat ramp-shaped spout 75 of the cover shell 70 to carry the diffused mist of aromatic fluid droplets to the outside open air in a substantially horizontal direction. Unlike conventional upward diffusion designs, the horizontal diffusion manner of the present invention enhances the effect of aroma diffusion.

Further, because the side block 315 is located at the periphery of the fluid container 30, it does not occupy any fluid accommodation space of the fluid container 30, and therefore the fluid accommodation capacity of the fluid container 30 is maximized.

Further, the air intake port 321 is configured to guide intake air toward the inside wall of the container cover 32 and the inside wall of the container body 31 to induce compact, enhancing the lifting of the mist of aromatic fluid droplets.

Referring to FIGS. 1 and 3 again, because the flat ramp-shaped spout 75, the coupling hole 72 and the coupling tube 322 are disposed in communication with one another, the diffused mist of aromatic fluid droplets can be forced out of the container body 31 of the fluid container 30 by the pressurized intake flow of air into the outside open air via the flat ramp-shaped spout 75, creating a visual effect.

Referring to FIG. 2 again, the rotating connection structure between the bottom holder shell 10 and the cover shell 70 facilitates general maintenance and cleaning activities. This rotating connection structure comprises a plurality of retaining studs 16 protruded from an inner side of the top edge 101 of the bottom holder shell 10, a bottom coupling flange 77 downwardly extended from the bottom side of the cover shell 70 in flush with the inside wall thereof, and a plurality of L-shaped retaining grooves 771 located at the bottom coupling flange 77. Each L-shaped retaining groove 771 has a short side 7710 vertically downwardly extended to the lowest edge of the bottom coupling flange 77.

The operation of the rotating connection structure between the bottom holder shell 10 and the cover shell 70 is explained hereinafter.

As shown in FIGS. 6-1, 6-2 and 6-3, when wishing to fasten the cover shell 70 to the bottom holder shell 10, aim the short sides 7710 of the L-shaped retaining grooves 771 at the respective retaining studs 16, and then insert the bottom coupling flange 77 of the cover shell 70 into the inside of the topmost edge of the bottom holder shell 10, and then rotate the cover shell 70 relative to the bottom holder shell 10 in clockwise direction to force the respective L-shaped retaining grooves 771 into engagement with the respective retaining studs 16. At this time, the cover shell 70 is locked to the bottom holder shell 10 and cannot be moved away from the bottom holder shell 10 in vertical direction.

As shown in FIG. 6-4, when wishing to dismount the bottom holder shell 10 and the cover shell 70, rotate the bottom holder shell 10 relative to the cover shell 70 in counter-clockwise direction to the extent to disengage the connection, allowing removal of the cover shell 70 from the bottom holder shell 10 directly.

In conclusion, the aromatic nebulizing diffuser of the present invention is characterized in that: the arrangement of the side block at the periphery of the fluid container and the formation of the pressurized space in between the cover shell and the fluid container enhance the effects of mist diffusion and maximize the fluid accommodation capacity; the design of the flat ramp-shaped spout of the cover shell enhances decorative and visual effects of aroma diffusion; the rotating connection structure between the bottom holder shell and the cover shell facilitates general maintenance and cleaning activities.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. An aromatic nebulizing diffuser, comprising:
a bottom holder shell comprising an accommodation open chamber and a plurality of air vents in communication with said accommodation open chamber;
an electric fan mounted in said accommodation open chamber and operable to suck outside air through said air vents into said accommodation open chamber;
a fluid container comprising a container body mounted in said bottom holder shell to close said accommodation open chamber and adapted for holding an aromatic fluid, said container body comprising a through hole located at a bottom side thereof and a side block located at the periphery thereof, said side block defining a bottom air inlet in air communication with said accommodation open chamber and said air vents of said bottom holder shell, a top air outlet and an air passage in communication between said bottom air inlet and said top air outlet, and a container cover detachably covering said container body, said container cover comprising an air intake port and a coupling tube in air communication with the inside space of said container body;
an oscillator sealed in said through hole of said container body of said fluid container and adapted to oscillate an aromatic fluid in said container body into a mist of aromatic fluid droplets;
a circuit board electrically coupled with said oscillator;
a power supply unit electrically connected to said electric fan, said oscillator and said circuit board to provide said electric fan, said oscillator and said circuit board with the necessary working voltage;
a cover shell comprising a bottom open end coupled to said bottom holder shell to surround said fluid container, a pressurized space defined therein around said fluid container, and an inside coupling hole coupled to said coupling tube.

2. The aromatic nebulizing diffuser as claimed in claim 1, wherein said bottom holder shell comprises a plurality of columns and a step located on the inside thereof; said container body comprises a bottom mounting flange supported on said step of said bottom holder shell and affixed to said columns with fastening members.

3. The aromatic nebulizing diffuser as claimed in claim 2, further comprising a gasket ring sealed in between said bottom mounting flange of said container body and said step of said bottom holder shell.

4. The aromatic nebulizing diffuser as claimed in claim 1, wherein said container cover comprises a plurality of locating rods; said cover shell comprises a plurality of locating sleeves respectively coupled to said locating rods of said container cover.

5. The aromatic nebulizing diffuser as claimed in claim 1, further comprising an oscillator sealing gasket ring mounted in said through hole of said container body of said fluid container to seal said oscillator in said through hole, and a locating plate bonded to said container body to seal said oscillator sealing gasket ring in said through hole of said container body.

6. The aromatic nebulizing diffuser as claimed in claim 1, wherein said cover shell further comprises a flat ramp-shaped spout located at a top side thereof in communication with said coupling hole for output of an oscillated mist of aromatic fluid droplets.

7. The aromatic nebulizing diffuser as claimed in claim 1, further comprising a plurality of elastic mounting members mounted in said accommodation open chamber of said bottom, and a plurality of light-emitting devices mounted in said elastic mounting members and electrically connected to said power supply unit.

8. The aromatic nebulizing diffuser as claimed in claim 1, wherein said bottom holder shell and said cover shell are selectively made in one of transparent, translucent, partially transparent, locally transparent, partially translucent and locally translucent forms.

9. The aromatic nebulizing diffuser as claimed in claim 1, further comprising a functional keypad mounted in said accommodation open chamber of said bottom holder shell, said functional keypad comprising a circuit board electrically coupled to said oscillator, said oscillator circuit board and said power supply unit, a plurality of key switches electrically connected to said circuit board for functional controls, and a plurality of light guide rods; said bottom holder shell comprises means for receiving said key switches and said light guide rods.

10. The aromatic nebulizing diffuser as claimed in claim 1, wherein said bottom holder shell comprises a plurality of retaining studs protruded from an inner side of a top edge thereof inside said accommodation open chamber; said cover shell comprises a bottom coupling flange downwardly extended from a bottom side thereof and insertable into said accommodation open chamber of said bottom holder shell, and a plurality of L-shaped retaining grooves located at said bottom coupling flange corresponding to said retaining studs, said L-shaped retaining grooves being respectively coupled to said retaining studs when said bottom coupling flange of said cover shell is vertically downwardly inserted into said accommodation open chamber of said bottom holder shell and then rotated with said cover shell horizontally relative to said bottom holder shell through a predetermined angle.
